(19) United States
(12) Patent Application Publication
(10) Pub. No.: US 2023/0009605 A1
(43) Pub. Date: Jan. 12, 2023

(54) INTEGRATED MICROFLUIDIC CHIP AND SINGLE-CELL CULTURE, SCREENING, AND EXPORT METHOD APPLYING SAME

(71) Applicant: Shanghai Industrial µTechnology Research Institute, Shanghai (CN)

(72) Inventors: Kun WANG, Shanghai (CN); Yimin GUAN, Shanghai (CN)

(73) Assignee: Shanghai Industrial µTechnology Research Institute, Shanghai (CN)

(21) Appl. No.: 17/863,372

(22) Filed: Jul. 12, 2022

(30) Foreign Application Priority Data
Jul. 12, 2021 (CN) 2021107854328

(51) Int. Cl. C12M 3/06; C12M 1/00; C12M 1/34; C12M 1/42

(52) U.S. Cl. CPC C12M 23/16; C12M 29/10; C12M 41/12; C12M 35/08

(57) ABSTRACT

An integrated microfluidic chip and a single-cell culture, screening, and export method applying the same are disclosed; the chip includes a base, an inlet flow channel, an outlet flow channel, a plurality of common flow channels and a plurality of functional units, wherein two ends of the common flow channel are connected to the inlet flow channel and the outlet flow channel, respectively, wherein each of the functional units includes a single-cell introduction port, a cell culturing-screening chamber, a cell export chamber, a cell export port, and a drive element, wherein the drive element is used to provide power to liquid to introduce single cells entering the common flow channels into the cell culturing-screening chamber, and after culturing and screening, to export target cell population in the cell culturing-screening chamber through the cell export port.

### CROSS REFERENCE TO RELATED APPLICATION

The present application claims the benefit of priority to Chinese Patent Application No. CN 202110785432.8, entitled “INTEGRATED MICROFLUIDIC CHIP AND SINGLE-CELL CULTURE, SCREENING, AND EXPORT METHOD APPLYING SAME”, filed with CNIPA on Jul. 12, 2021, the disclosure of which is incorporated herein by reference in its entirety for all purposes.

### FIELD OF TECHNOLOGY

The present disclosure generally relates to the field of microfluidics, cell line development, and monoclonal antibody screening, in particular, to an integrated microfluidic chip, and a single-cell culture, screening, and export method applying the same.

### BACKGROUND

A cell is the basic unit of life activity, and research based on single cells can reveal the development pattern of life activity at a deeper level and therefore has wide application in the fields of monoclonal antibody screening, cell line culture, etc. Single-cell isolation is the foundation and key of single-cell research. Currently, single-cell isolation is mainly performed by micro-needle aspiration, finite dilution, micro-well arrays, and microfluidic-based sorting methods. Current isolation methods face problems of difficult operation, low efficiency, and multi-cell acquisition, which are not conducive to subsequent culture and analysis. In monoclonal antibody screening and cell line culture applications, isolated single cells are mostly placed in well plates for culture, and after potency and phenotype analysis, the cell populations are screened to obtain cell populations that perform well for large-scale culture. The whole process is labor-intensive, tedious, time-consuming, and inefficient. Therefore, in single-cell research, especially monoclonal antibody screening and cell line development, there is an urgent need for an efficient research method that is simple to operate and integrates single cell isolation, culture, screening, and export.

### SUMMARY

The present disclosure provides an integrated microfluidic chip, including: a base, including a front side and a back side set opposite to each other; an inlet flow channel and an outlet flow channel, both buried in the base and spaced apart; a plurality of common flow channels, buried in the base and spaced apart, wherein each of the common flow channels have two ends connected to the inlet flow channel and the outlet flow channel, respectively; and a plurality of functional units, each of the functional units including a single-cell introduction port, a cell culturing-screening chamber, a cell export chamber, a cell export port, and a drive element, wherein the single-cell introduction port is provided on the front side of the base and connected to a first common flow channel of the common flow channels, wherein the cell culturing-screening chamber and the cell export chamber are both buried in the base, and both ends of the cell culturing-screening chamber are connected to the first common flow channel and the cell export chamber, wherein the cell export port is provided at the back side of the base and is connected to the cell export chamber, respectively, wherein the drive element is located at an inner surface of the cell export chamber away from the back side of the base and faces the cell export port, wherein the drive element is used to provide power to liquid for introducing single cells entering the common flow channel into the cell culturing-screening chamber and, after culturing and screening, to export target cell population in the cell culturing-screening chamber through the cell export port.

Optionally, the inlet flow channel extends in a direction parallel to a direction in which the outlet flow channel extends.

Optionally, the common flow channels extend in a direction perpendicular to a direction in which the inlet flow channel extends.

Optionally, a thickness of the cell culturing-screening chamber is so set that the cell culturing-screening chamber accommodates only a single layer of cells.

Optionally, a width of the cell culturing-screening chamber is greater than a width of the cell export chamber, wherein the two widths are in a direction perpendicular to the direction pointing from the cell culturing-screening chamber towards the cell export chamber.

Optionally, the single-cell introduction port is used to receive single cells ejected from a single-cell printing chip.

Optionally, the single-cell printing chip includes a thermal bubble printing chip.

Optionally, the drive element includes one of a heating film, a PDMS microvalve, a solenoid valve, and a peristaltic pump.

Optionally, the number of the functional units is in the range of 10 to 10000.

The present disclosure further provides a single-cell culture, screening, and export method, including: providing an integrated microfluidic chip as described above, and injecting single cells by the single-cell introduction port into one of the common flow channels, i.e., the first common flow channel; after allowing the single cells to settle naturally, introducing the single cells injected into the first common flow channel into the cell culturing-screening chamber by using the drive element to drive liquid to flow; after the single cells have been cultured in the cell culturing-screening chamber for a predetermined time, introducing a screening reagent into the cell culturing-screening chamber via the inlet flow channel to identify and obtain target cell populations; and transferring the target cell populations to a designated container through the cell export port.

Optionally, before injecting the single cells by the single-cell introduction port into the first common flow channel, cell culture fluid is first perfused into the integrated microfluidic chip to drain air bubbles.

Optionally, after introducing the single cells injected into the first common flow channel into the cell culturing-screening chamber, the cell culture fluid is again perfused to culture cells.

Optionally, after introducing the screening reagent into the cell culturing-screening chamber, cell populations in the cell culturing-screening chamber are screened to obtain the target cell populations by image characterization.

Optionally, the single-cell culture, screening, and export method is used to perform screening to obtain monoclonal antibody cell populations

As described above, the present disclosure provides an integrated microfluidic chip for isolation and culture of single-cells, and screening and export of target cell populations, which enables single-cell culture, cell population identification and screening, and target cell population export to be done on a chip, improving cell throughput, simplifying experimental operations, and reducing reagent usage and cross-contamination risks. Meanwhile, the whole process can be made more controllable, convenient and efficient when combined with thermal bubble printing technology..

### BRIEF DESCRIPTION OF THE DRAWINGS

FIG. 1 shows a top view of a structure of an integrated microfluidic chip of the present disclosure.

FIG. 2 shows a side view of a structure of a functional unit in an integrated microfluidic chip of the present disclosure.

FIG. 3 shows a flow chart of a single-cell culture, screening, and export method of the present disclosure.

### REFERENCE NUMERALS

1 Base

2 Inlet flow channel

3 Outlet flow channel

4 Common flow channel

5 Functional unit

501 Single-cell introduction port

502 Cell culturing-screening chamber

503 Cell export chamber

504 Cell export port

505 Drive element

6 Single Cell

7 Single-cell printing chip

S1˜S4 Various steps

### DETAILED DESCRIPTION

The following describes the implementation of the present disclosure through specific examples, and those skilled in the art can easily understand other advantages and effects of the present disclosure from the content disclosed in this specification. The present disclosure can also be implemented or applied through other different specific embodiments. Various details in this specification can also be modified or changed based on different viewpoints and applications without departing from the spirit of the present disclosure.

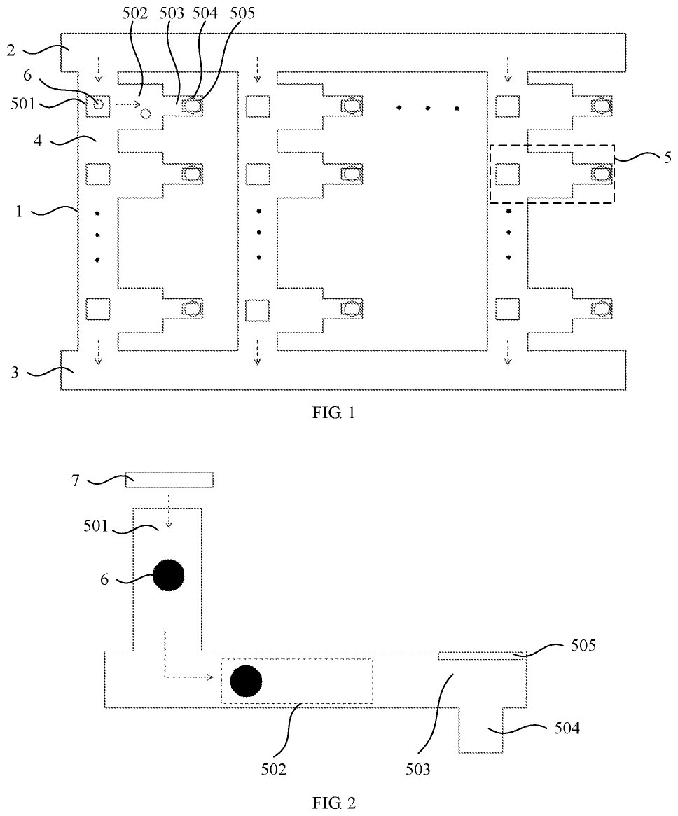

Refer to FIGS. 1 to 3. It should be noted that the drawings provided in this disclosure only illustrate the basic concept of the present disclosure in a schematic way, so the drawings only show the components related to the present disclosure. The drawings are not necessarily drawn according to the number, shape and size of the components in actual implementation; during the actual implementation, the type, quantity and proportion of each component can be changed as needed, and the components' layout may also be more complicated.

### Embodiment 1

In this embodiment, an integrated microfluidic chip is provided. Referring to FIG. 1, a top view of the integrated microfluidic chip is shown, including a base 1, an inlet flow channel 2, an outlet flow channel 3, a plurality of common flow channels 4, and a plurality of functional units 5. Referring to FIG. 2, a side view of one of the functional units 5 is shown.

Specifically, the base 1 includes a front side and a back side set opposite to each other; the inlet flow channel 2 and the outlet flow channel 3 are buried in the base 1 and spaced apart; the plurality of the common flow channels 4 are buried in the base 1 and spaced apart from each other, and two ends of each of the common flow channels 4 are connected to the inlet flow channel 2 and the outlet flow channel 3, respectively; the plurality of the functional units 5 are arranged in an array, and each of the functional units 5 includes a single-cell introduction port 501, a cell culturing-screening chamber 502, a cell export chamber 503, a cell export port 504, and a drive element 505, wherein the single-cell introduction port 501 is provided on the front side of the base 1 and connected to one of the common flow channels 4, namely a first common flow channel 4, wherein the cell culturing-screening chamber 502 and the cell export chamber 503 are buried in the base 501, wherein both ends of the cell culturing-screening chamber 502 are connected to the first common flow channel 4 and the cell export chamber 503, respectively, wherein the cell export port 504 is provided at the back side of the base 1 and is connected to the cell export chamber 503, wherein the drive element 505 is located at an inner surface of the cell export chamber 503 away from the back side of the base 1 and faces the cell export port 504, and the drive element 505 is used to provide power for liquid to introduce single cells 6 entering the first common flow channel 4 into the cell culturing-screening chamber 502, and after culturing and screening, to export target cell populations in the cell culturing-screening chamber 502 through the cell export port 504.

Specifically, the functional units 5 have functions of single cell separation, culture, screening, and export, and thousands of the functional units 5 can be integrated in the integrated microfluidic chip as needed to ensure high throughput of single-cell culture and screening. As an example, the number of the functional units 5 ranges from 10 to 10,000, e.g. 1000, 2000, 5000, etc.

As an example, flow paths of liquid are illustrated in FIG. 1 using dashed arrows. The inlet of the inlet flow channel 2 and the outlet of the outlet flow channel 3 can be adjusted as desired; e.g. they can be located at one end of the flow channel or at a predetermined position in the middle of the flow channel, the latter of which is not illustrated in the figures. Culture fluids and reagents can be driven by external power for the culture and screening of cells.

As an example, as shown in FIG. 1, the inlet flow channel 2 extends in a direction parallel to the direction in which the outlet flow channel 3 extends, and the common flow channels 4 extend in a direction perpendicular to the direction in which the inlet flow channel 2 extends, so that a plurality of the functional units 5 are arranged in a regular quadrilateral array, which makes the layout compatible with printing nozzles of a single-cell printing chip and also with a cell receiving device. In other embodiments, the inlet flow channel 2, the outlet flow channel 3, and the common flow channels 4 are connected in other manners that can also be adjusted as needed.

As an example, as shown in FIG. 2, the thickness of the cell culturing-screening chamber 502 is so set that the chamber accommodates only a single layer of cells; for example, the thickness of the cell culturing-screening chamber 502 may be set to be comparable to the size of the cells to be screened, thereby ensuring a single layer of cells to be arranged during cell culture, which helps subsequent cell counting and fluorescence analysis.

As an example, as shown in FIG. 1, the width of the cell culturing-screening chamber 502 is greater than the width of the cell export chamber 503, with both widths in a direction perpendicular to the direction pointing from the cell culturing-screening chamber 502 towards the cell export chamber 503, so that the target cells can sequentially enter into the cell export chamber 503 and then be exported.

In an example, the drive element 505 may include one of a heating film, a polydimethylsiloxane (PDMS) micro-valve, a solenoid valve, and a peristaltic pump. In this embodiment, the drive element 505 includes preferably a heating film so as to form a thermal bubble nozzle along with the cell export chamber 503 and the cell export port 504. The thermal bubble nozzle is made based on a micro-nano processing process and is integrated in a bottom of the micro-channels. The thermal bubble nozzle utilizes instantaneous high temperature of the heating film to vaporize liquid at the top area of the cell export chamber 503, generating bubbles to propel the flow of liquid and eject the liquid from the nozzle, followed by subsequent liquid replenishment by capillary forces, thus providing power for the continuous flow of the liquid. The thermal bubble nozzle is controlled by an underlying circuitry.

As an example, as shown in FIG. 2, the single-cell introduction port 501 is used to receive single cells 6 ejected from the single-cell printing chip 7, and the single-cell introduction port 501 is positioned such that when the single cells enter the common flow channels through the single-cell introduction port 501, sedimentation positions of the single cells are located in an inlet area of the cell culturing-screening chamber 502, so that under the driving action of the drive element 505, the single cells 6 can then accurately enter the cell culturing-screening chamber 502. The single-cell printing chip 7 can be a single-cell printing chip as disclosed in Chinese patent No. CN108330065A or other suitable single-cell printing chips to achieve separation of single cells and introduction thereof into the cell introduction port 501. A single-cell printing chip including a thermal bubble printing chip is preferred in this embodiment, which is less harmful to cells and can gently introduce single cells into the single-cell introduction port 501 with high efficiency.

The integrated microfluidic chip of the present disclosure can be used for single cell isolation, culture, and screening and export of target cell population, so that single-cell culture, cell population identification and screening, and target cell population export are all done on the chip, which improves cell throughput, simplifies experimental operations, and reduces reagent usage and cross-contamination risks. Meanwhile, the whole process can be made more controllable, convenient and efficient when combined with thermal bubble printing technology.

### Embodiment 2

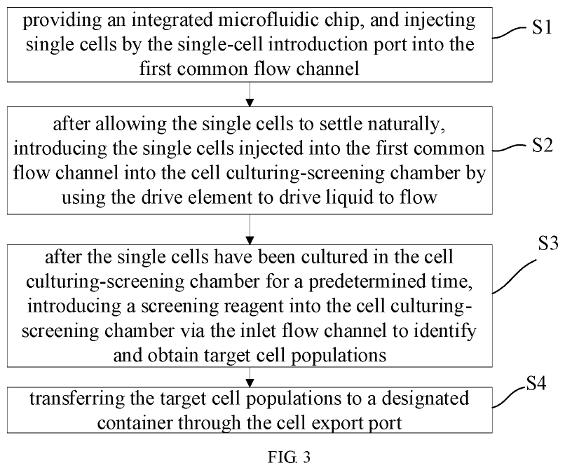

A single-cell culture, screening, and export method is provided in this embodiment, as shown in FIG. 3, which is a flow chart of the method, including:

S1: providing an integrated microfluidic chip as described above, and injecting single cells by the single-cell introduction port into one of the common flow channels, i.e., a first common flow channel;

S2: after allowing the single cells to settle naturally, introducing the single cells injected into the first common flow channel into the cell culturing-screening chamber by using the drive element to drive liquid to flow;

S3: after the single cells have been cultured in the cell culturing-screening chamber for a predetermined time, introducing a screening reagent into the cell culturing-screening chamber via the inlet flow channel to identify and obtain target cell populations; and

S4: transferring the target cell populations to a designated container through the cell export port;

As an example, in the step S1, before injecting the single cells from the single-cell introduction port into the first common flow channel, cell culture fluid is first perfused into the integrated microfluidic chip to drain air bubbles.

As an example, in the step S2, after introducing the single cells injected into the first common flow channel into the cell culturing-screening chamber, the cell culture fluid is again perfused into the integrated microfluidic chip to culture cells.

As an example, in the step S3, after introducing the screening reagent into the cell culturing-screening chamber, cell populations in the cell culturing-screening chamber are screened to obtain the target cell populations by image characterization. The screening reagent may be a phenotypic identification antibody or other target, depending on the cells to be screened for.

As an example, in the step S4, preferably, the target cell populations are transferred to a designated container for subsequent culture or analysis using a thermal bubble printing method. The thermal bubble printing method has advantages such as fast response, strong driving force, easy control, easy integration and miniaturization, which ensures that the whole process be carried out conveniently and efficiently.

As an example, the single-cell culture, screening, and export method of this embodiment can be used to perform screening to obtain monoclonal antibody cell populations or other types of cell populations.

### Embodiment 3

This embodiment uses the microfluidic chip of embodiment 1 for monoclonal cell line screening. First, the cell culture fluid is perfused into the chip and air bubbles are expelled. Then perfusion of the cell culture fluid stops and transfected cells are passed through the single-cell printing chip and placed as individual cells in the single-cell introduction port of the chip. The thermal bubble nozzle is controlled by the underlying circuitry to bring single cells into the cell culturing-screening chamber. To obtain cell populations arranged in a single layer, the height of the cell culturing-screening chamber can be adjusted according to the size of the cells used. After the cell culture fluid has been reperfused and cells have been cultured to reach a certain number of populations, relevant fluorescent antibodies are injected at a reagent inlet to characterize potency of antibodies secreted by each cell population. The target cell populations are then obtained after screening, as needed. At this point, the target cell populations are transferred to a designated container through the thermal bubble nozzle at the cell export port to complete the screening for monoclonal antibody cell populations.

In summary, the present disclosure provides an integrated microfluidic chip for isolation and culture of single-cells, and screening and export of target cell populations, which enables single-cell culture, cell population identification and screening, and target cell population export to be done on a chip, improving cell throughput, simplifying experimental operations, and reducing reagent usage and cross-contamination risks. Meanwhile, the whole process can be made more controllable, convenient and efficient when combined with thermal bubble printing technology. Therefore, the present disclosure effectively overcomes various shortcomings of the prior art and has a high value for industrial application.

The above-mentioned embodiments only exemplarily illustrate the principles and effects of the present disclosure, but are not used to limit the present disclosure. Any person skilled in the art may modify or change the above embodiments without violating the spirit and scope of the present disclosure. Therefore, all equivalent modifications or changes made by those skilled in the art without departing from the spirit and technical concepts disclosed by the present disclosure should still be covered by the attached claims of the present disclosure.

What is claimed is:

1. An integrated microfluidic chip, comprising:
a base, comprising a front side and a back side set opposite to each other;
an inlet flow channel and an outlet flow channel, both buried in the base and spaced apart;
a plurality of common flow channels, buried in the base and spaced apart, wherein each of the common flow channels has two ends connected to the inlet flow channel and the outlet flow channel, respectively; and
a plurality of functional units, wherein each of the functional units comprises a single-cell introduction port, a cell culturing-screening chamber, a cell export chamber, a cell export port, and a drive element, wherein the single-cell introduction port is provided on the front side of the base and connected to a first common flow channel of the common flow channels, wherein the cell culturing-screening chamber and the cell export chamber are both buried in the base, and both ends of the cell culturing-screening chamber are connected to the common flow channel and the cell export chamber, respectively, wherein the cell export port is provided at the back side of the base and is connected to the cell export chamber, wherein the drive element is located at an inner surface of the cell export chamber away from the back side of the base and faces the cell export port, wherein the drive element is used to provide power to liquid for introducing single cells entering the common flow channel into the cell culturing-screening chamber and, after culturing and screening, to export target cell population in the cell culturing-screening chamber through the cell export port.

2. The integrated microfluidic chip according to claim 1, wherein the inlet flow channel extends in a direction parallel to a direction in which the outlet flow channel extends.

3. The integrated microfluidic chip according to claim 2, wherein the common flow channels extend in a direction perpendicular to a direction in which the inlet flow channel extends.

4. The integrated microfluidic chip according to claim 1, wherein a thickness of the cell culturing-screening chamber is so set that the cell culturing-screening chamber accommodates only a single layer of cells.

5. The integrated microfluidic chip according to claim 1, wherein a width of the cell culturing-screening chamber is greater than a width of the cell export chamber, wherein the two widths are in a direction perpendicular to the direction pointing from the cell culturing-screening chamber towards the cell export chamber.

6. The integrated microfluidic chip according to claim 1, wherein the single-cell introduction port is used to receive single cells ejected from a single-cell printing chip.

7. The integrated microfluidic chip according to claim 6, wherein the single-cell printing chip comprises a thermal bubble printing chip.

8. The integrated microfluidic chip according to claim 1, wherein the drive element comprises one of a heating film, a PDMS micro-valve, a solenoid valve, and a peristaltic pump.

9. The integrated microfluidic chip according to claim 1, wherein the number of the functional units is in the range of 10 to 10000.

10. A method for single-cell culture, screening, and export, comprising:
providing an integrated microfluidic chip as claimed in claim 1, and injecting single cells by the single-cell introduction port into the first common flow channel;
after allowing the single cells to settle naturally, introducing the single cells injected into the first common flow channel into the cell culturing-screening chamber by using the drive element to drive liquid to flow;
after the single cells have been cultured in the cell culturing-screening chamber for a predetermined time, introducing a screening reagent into the cell culturing-screening chamber via the inlet flow channel to identify and obtain target cell populations; and
transferring the target cell populations to a designated container through the cell export port.

11. The single-cell culture, screening, and export method according to claim 10, wherein before injecting the single cells by the single-cell introduction port into the first common flow channel, cell culture fluid is first perfused into the integrated microfluidic chip to drain air bubbles.

12. The single-cell culture, screening, and export method according to claim 11, wherein after introducing the single cells injected into the first common flow channel into the cell culturing-screening chamber, the cell culture fluid is again perfused to culture cells.

13. The single-cell culture, screening, and export method according to claim 10, wherein after introducing the screening reagent into the cell culturing-screening chamber, cell populations in the cell culturing-screening chamber are screened to obtain the target cell populations by image characterization.

14. The single-cell culture, screening, and export method according to claim 10, wherein the single-cell culture, screening, and export method is used to perform screening to obtain monoclonal antibody cell populations.
